(19) United States
(12) Patent Application Publication
(10) Pub. No.: US 2017/0196517 A1
(43) Pub. Date: Jul. 13, 2017

(54) METHOD AND DEVICE FOR DETERMINING THE CENTRAL SYSTOLIC BLOOD PRESSURE

(71) Applicant: FRESENIUS MEDICAL CARE DEUTSCHLAND GMBH, Bad Homburg (DE)

(72) Inventor: Wei ZHANG, Niederwerrn (DE)

(73) Assignee: FRESENIUS MEDICAL CARE DEUTSCHLAND GMBH, Bad Homburg (DE)

(21) Appl. No.: 15/313,462

(22) PCT Filed: May 22, 2015
(86) PCT No.: PCT/EP2015/061371

§ 371 Date: Nov. 22, 2016

(30) Foreign Application Priority Data
May 22, 2014 (DE) 10 2014 007 519.2

(51) Int. Cl. A61B 5/00; A61B 5/023; A61B 5/021

(52) U.S. Cl. CPC A61B 5/725; A61B 5/02108; A61B 5/023

(57) ABSTRACT

The invention relates to a method for determining the central systolic blood pressure of a patient, comprising the following steps:
determining a peripheral, in particular brachial, blood pressure curve,
applying a moving average filter to the blood pressure curve,
applying an all-pass filter to the blood pressure curve,
determining the central systolic blood pressure as the maximum of the all-pass-filtered and average-filtered blood pressure curves, as well as a device for performing this method.

The invention relates to a device for determining the central systolic blood pressure, a blood treatment machine and a method for determining the central systolic blood pressure.

In dialysis patients, cardiovascular diseases are the main cause of death. Severe vascular calcification and, associated with that, a stiffening of the central blood vessels (in particular the aorta) play a major role in this period of time in particular. For this reason, there is a great deal of interest in monitoring the central vascular stiffness in dialysis patients.

The pulse wave velocity (PWV) can be measured and evaluated as an indicator of vascular stiffness because a pulse wave travels more rapidly in a blood vessel as the stiffness increases.

The central blood pressure level is another important characteristic number for detecting cardiovascular diseases. The peripheral blood pressure, measured traditionally by a cuff on the upper arm, for example, hardly provides any information about the central blood pressure level, because calcification of the central vessels proceeds much more rapidly than that of the peripheral blood vessels, among other things. The actual central blood pressure can be determined definitively by inserting a catheter into the ascending aorta and then using the catheter to measure the aortic blood pressure. Such invasive measurements have become the “gold standard,” but in the patient's interests, they can by no means be performed routinely just to observe the trend in the central blood pressure over the long term.

Karamanoglu et al. (“An analysis of the relationship between central aortic and peripheral upper limb pressure waves in man,” European Heart Journal 14, 160-167, 1993) therefore proposed transforming a brachial pressure pulse curve, i.e., a measured curve of the bloOd pressure measured on the upper arm, to a central pulse curve by way of a so-called generalized transfer function (GTF). The generalized transfer function was determined by measurements on a test subject population, in which the actual aortic blood pressure was determined invasively and then a function, which converts the pressure pulse curve measured brachially to the measured central pressure pulse curve, was calculated. One disadvantage here is that the transfer requires the time-resolved measurement signals to be converted into frequency domains, namely transformation by means of the transfer function and a back-transformation of the resulting signals into time domains. This involves a complex signal processing that requires corresponding computation capacities, which cannot be made available within the context of a dialysis treatment or can be made available only with great effort and at great expense.

The object of the present invention is to overcome the aforementioned disadvantages of the prior art and to propose a method by which the central systolic blood pressure can be estimated with little equipment and a minor computational outlay. Another object is to provide a device with which the central systolic blood pressure can be estimated as well as to provide a blood treatment machine containing such a device.

This object is achieved with a method according to claim 1. According to this claim, a method for determining the central systolic blood pressure of a patient comprises the following steps:
determining a peripheral, in particular brachial, blood pressure curve over time,
applying a moving average filter to the blood pressure curve,
applying an all-pass filter to the blood pressure curve,
determining the central systolic blood pressure as the maximum filtered blood pressure curve, i.e., the blood pressure curve to which a moving average filter and an all-pass filter have been applied.

A moving average filter may be a low-pass filter, in which filtered signal values are formed by averaging a group of signal values occurring before and after the respective measuring point—i.e., in the present invention, the peripheral pressure values, plotted as a function of time thus determined. This time window for a group of signal values of the peripheral blood pressure curve is moved successively on the time axis for calculation of the filtered blood pressure curve.

The method according to the invention has the advantage that the central systolic blood pressure can be determined by a noninvasive method using very simple means. To do so, it is not necessary to perform any complex computation methods such as Fourier transforms, so that only a low computation capacity and short time are required. Furthermore, only the group of signal values required for the respective average signal value must be stored temporarily to form the average in each case. As soon as the average has been determined from this group of signal values, this group of signal values can be overwritten by the next group. This makes it possible to perform the calculation in conventional commercial microcontrollers, for example. Details of how the computation capacity is utilized can be decided by the skilled person, so that the aforementioned procedure for overwriting is intended only as an example and is not to be regarded as restricting the disclosure content of the invention.

According to one aspect of the method, the peripheral blood pressure curve can be determined first as a relative curve and the patient's systolic pressure and diastolic pressure can be determined as absolute values. An absolute peripheral blood pressure curve can be determined by calibration of the relative blood pressure curve determined using the systolic and diastolic pressure values.

The relative peripheral blood pressure curve, the systolic pressure, the diastolic pressure and/or the patient's pulse can be measured oscillometrically. This makes it possible to determine the blood pressure curve and the aforementioned values using a traditional arm cuff for determining the brachial blood pressure by applying the cuff to the upper arm. This determination may be made if a traditional blood pressure measurement is performed on the patient, for example. This may be done as part of a regular treatment, such as an extracorporeal blood treatment, for example. This has the advantage that regular monitoring of the central systolic blood pressure is possible because the measurement can be performed with very little effort.

The pulse can be given as heart rate HR (in bpm, i.e., beats per minute) or as the heart rate [frequency] f_{HR} (in Hz).

The blood pressure cuff or the like may be used in general to achieve hemostasis in a vein. Therefore, puncturing the vein for an infusion, for example, can be facilitated. It is possible to provide for the blood pressure cuff to be pumped up to a predetermined pressure. The pumping operation may be performed by means of an operating device such as a button, for example.

According to a preferred variant of the process, the moving average filter is applied to the blood pressure curve in such a way that a fraction of the amount of the scanning frequency for measuring the peripheral blood pressure curve with which the signal values are recorded is selected as the window width of a time window of a group of measuring points of the peripheral blood pressure curve. The denominator to form the fraction may be between 3 and 5. The denominator may be between 3.8 and 4.2 in particular, for example, approximately 4 or exactly 4. In comparison with values actually measured by the invasive method, particularly accurate results in the numerical range are obtained for the central systolic blood pressure. When the patient's pulse is 70 bpm, denominator values between 3.8 and 4.2, such as approximately 4 or precisely 4, yield especially accurate values for the systolic blood pressure. The scanning frequency, which is divided by the denominator n, is given in hertz (Hz). It may be advantageous if the denominator n is an integer, but this is not a prerequisite for this variant of the process. The resulting fractions of the sampling frequency and the denominator n are advantageously rounded off to integers.

According to one variant of the process, the time window is created with a window width of 2m+1 on the time axis about a respective measuring point of the peripheral blood pressure curve, where m is an integral variable for determining a number of measuring points. The window here is advantageously drawn symmetrically with the respective measuring point, such that there are m neighboring measuring points to the right and to the left of the measuring point, forming the group of measuring points, of which the respective average is formed together with the specific measuring point itself.

The all-pass filter can be applied by adding an offset to the average-filtered blood pressure curve.

The filtered blood pressure curve y(k) is advantageously determined according to a filter equation (1):

$$y ( k ) = A \cdot x ( k ) + B \cdot \frac{1}{2 m + 1} \cdot {\sum }_{i = - m}^{m} x ( k + i ) \qquad ( 1 )$$

The offset can be expressed by an all-pass coefficient A in conjunction with the absolute peripheral blood pressure curve x(k); B is an average filter coefficient, where A+B=1, and k is the index of the measuring points of the peripheral blood pressure curve, and i is the index of neighboring measuring points for the determination of the average. A filtered blood pressure curve y(k), which is suitable for determining the central systolic blood pressure as its maximum, can be calculated very easily with equation (1).

The all-pass coefficient A, the average filter coefficient B and the window width N can preferably be determined in particular by the fact that a transfer function, which corresponds to the filter equation, is approximated to a calibration transfer function.

This permits an approximation to a calibration transfer function for transfer of a peripheral blood pressure curve to a central systolic blood pressure curve, of which it is advantageously known that it reproduces the actual systolic blood pressure curve of the aorta especially accurately.

A and B may be between 0.2 and 0.8, for example, in particular between 0.3 and 0.7, in particular between 0.4 and 0.6. They may each have a value of 0.5, For example.

The transfer function of equation (1) can be expressed with equation (2), for example:

$$H ( f ) = A + \frac{B}{N} \cdot \frac{sin ( π Nf / {f}_{s} )}{sin ( π f / {f}_{s} )} \qquad ( 2 )$$

where f is a frequency, f_{S} is the sampling frequency and N(=2m+1) is the window width. The transfer function is represented in the frequency domain instead of in the time domain like the filtered blood pressure curve.

According to a first variant, the calibration transfer function may be a generalized transfer function (GTF). Such a generalized transfer function can be determined from measurements on a population of patients and/or volunteers who do not necessarily correspond to or match the patient himself. A curve for the central systolic blood pressure can be measured invasively based on this patient/volunteer population. An averaged overall curve can be compiled as a calibration blood pressure curve from the individual central blood pressure curves measured on the patient/volunteer population. This can be compared with a peripheral systolic blood pressure curve for the same patient/volunteer population and one can be converted to the other by means of a generalized transfer function yet to be determined. This has the advantage that it is not necessary to perform an invasive procedure on the patient. The installation of a cardiac catheter which would be necessary for that can thus be avoided. It may be necessary to determine the generalized transfer function with a relatively high computation effort, but it is then available as a calibration transfer function for the method according to the invention without having to recalculate it again each time for application of the method according to the invention.

In addition, there is the possibility of accessing generalized transfer functions as calibration transfer functions, which are already available because they are known from the literature, for example. This reduces the effort for obtaining a calibration transfer function.

According to another variant, the calibration transfer function of a central systolic blood pressure curve may be determined as a calibration blood pressure curve which is measured invasively, in particular on the ascending aorta and/or the patient's aortic root. This ensures that the actual central systolic blood pressure curve and thus also the central systolic blood pressure as its maximum are known.

This calibration pressure curve per se fulfills the property that influences such as age, disease or dosing are taken into account. This is advantageous in particular when a catheter examination is already being performed on the patient anyway, so that no additional procedure is necessary. In this variant, the individual patient may be considered as a special case of a patient population.

It may be preferable to correct the window width using a pulse-dependent correction factor (i.e., depending on the heart rate, i.e., heart frequency) to a corrected window width. Therefore, especially good values for the central systolic blood pressure can be achieved even with pulse values, which deviate up or down from a heart rate in the usual range. In the case of heart rates that are less than 50 bpm or more than 90 bpm in particular, a correction with a pulse-dependent correction factor is advantageous. However, a pulse-dependent correction may be advantageous even at less than 60 bpm or more than 80 bpm. However, it may also be advantageous to perform a correction in the vicinity of a pulse of 70 bpm, for example, at 65 or 75 bpm.

According to one variant, the pulse-corrected window width is calculated as the absolute value of a quotient of the scanning frequency and the pulse-corrected denominator. Then the pulse-corrected denominator is itself calculated by multiplying the denominator from the determination of the window width for the patient population, which has a reference heart rate HR_{Ref} times the quotient of the patient's current heart rate and the reference heart rate HR_{Ref}. The patient's current heart rate is understood in particular to be the heart rate prevailing in the determination of the peripheral, in particular brachial, blood pressure curve over time. The reference heart rate is the same heart rate HR_{Ref} as that found in determination of the calibration pressure curve. The calculated average heart rate of the patient population in measurement of the central and peripheral blood pressure curve is referred to here as the heart rate of the patient population. This can be expressed by equation (3), in which f_{S} is the scanning frequency in hertz, HR_{Pat} is the current heart rate, HR_{Ref} is the reference heart rate, n is the denominator and k is a correction factor amounting to 1, for example, but it may also be between 0.5 and 1.5, depending on the required corrections.

$$N = \vert \frac{{f}_{s}}{nk} \cdot \frac{1}{{HR}_{pat} / {HR}_{Ref}} \vert \qquad ( 3 )$$

According to another aspect of the invention, the filtered blood pressure curve may contain a phase shift of the average filter, which can be represented in equation (4), which supplements equation (2) by adding a phase angle φ.

$$y ( k ) - A \cdot x ( k ) + B \cdot \frac{1}{2 m + 1} \cdot {\sum }_{i = - m}^{m} x ( k + i - ϕ ) \qquad ( 4 )$$

Thus the precise curve form of the central blood pressure curve and additional cardiovascular characteristic values may advantageously be determined therefrom. The maximum of the simple systolic blood pressure curve determined according to the invention as the filtered blood pressure curve is advantageously the same with and without taking into account the phase angle.

In one variant of the invention, the transfer function corresponding to equation (4) can be expressed by taking into account the phase angle through equation (5). Thus the all-pass coefficient A, the average filter coefficient B, the window width N and/or the denominator n can be determined by including the phase angle φ.

$$H ( f ) = A + \frac{B}{N} \cdot \frac{sin ( π Nf / {f}_{s} )}{sin ( π f / {f}_{s} )} * {e}^{- j 2 πϕ f / {f}_{s}} \qquad ( 5 )$$

The object of the present invention is additionally achieved with a device according to claim 12. Such a device for determining the central systolic blood pressure of the patient has a blood pressure measuring device for determining the peripheral, in particular brachial, blood pressure curve as well as an evaluation unit, which is configured for
applying a moving average filter to the blood pressure curve,
applying an all-pass filter to the blood pressure curve,
determining the central systolic blood pressure as the maximum of the filtered blood pressure curve.

The central systolic blood pressure of a patient can be determined by simple technical means using the device according to the invention. To apply a moving average filter and an all-pass filter, only a low computation power and low storage capacity are required. The blood pressure measuring device may be a traditional blood pressure measuring device for measuring the peripheral blood pressure, in particular the brachial blood pressure. The blood pressure measuring device can record the peripheral blood pressure curve itself or the evaluation unit can determine the peripheral blood pressure curve by individual measured values of the blood pressure measuring device to form one curve. This is not relevant for the invention. For the sake of simplicity, the two variants are combined with the formulation that the blood pressure measuring device is provided for determining the peripheral blood pressure curve.

According to one aspect of the present invention, the blood pressure measuring device may be suitable for determining the peripheral blood pressure curve as a relative value set and determining the systolic and diastolic blood pressure of the patient as absolute values. According to another aspect, the evaluation unit may be configured to determine an absolute peripheral blood pressure curve by calibration of the relative blood pressure curve with the absolute systolic and diastolic blood pressure. Alternatively, the blood pressure measuring device itself may have the equipment necessary for determining an absolute peripheral blood pressure curve by calibration of the relative blood pressure curve with the absolute systolic and diastolic blood pressure curves.

The evaluation unit is advantageously configured to perform the method according to the invention in one of the embodiments or alternatives described above or to perform a combination of several of the embodiments described or alternatives or all of the embodiments or alternatives of the method according to the invention, as described here.

A transfer function or a calibration transfer function, an all-pass coefficient A, an average filter coefficient B, a window width N and/or a denominator n is/are advantageously stored in the evaluation unit so that they can be accessed when determining the central systolic blood pressure. This stored data may be available on a so-called patient card, for example, i.e., a memory card that is connected to the evaluation unit and/or can access the evaluation unit.

It may also be advantageous for the evaluation unit to have a computation unit, which is configured to perform process steps, including calculation of a transfer function or a calibration transfer function, determination of the all-pass coefficient A, the average filter coefficient B, the window width N and/or the denominator n.

The computation unit may advantageously be available as a separate part of the evaluation unit. By separating the computation unit from the remaining evaluation unit, it is advantageously possible to achieve the result that the evaluation unit is equipped with a computation capacity and a memory volume, which must be designed to be only large enough to perform the filtering of the peripheral blood pressure curve but need not be suitable for more complex calculations that must be performed rarely, for example, only once in the course of a measurement series lasting several years.

The object of the present invention is additionally achieved with a blood treatment machine according to claim 15. Accordingly, the blood treatment machine has a device according to the invention for determining the central systolic blood pressure of patient. This makes it possible for the central systolic blood pressure to also be determined at this opportunity on patients who are being treated with a blood treatment machine, for example, in addition to performing a traditional brachial blood pressure measurement. In the case of a regular blood treatment, the central systolic blood pressure can also be determined regularly at this opportunity. The blood treatment machine may be a hemodialysis machine, a hemofiltration machine or a hemodiafiltration machine, for example.

According to one variant of the blood treatment machine, the machine for determining a patient's central systolic blood pressure is combined with a machine for measuring and/or monitoring the blood pressure of the patient being treated by the blood treatment machine, wherein the machine for measuring and/or monitoring the blood pressure is assigned to the blood treatment machine.

In addition, the device for determining a patient's central systolic blood pressure may be combined with a machine for determining the stiffness of the blood vessels. With this machine, the pulse wave velocity may be measured and evaluated as an indicator of the vascular stiffness. Together with the vascular stiffness, the value for the central systolic blood pressure permits even more accurate information to be derived about any cardiovascular diseases. The evaluation unit may advantageously be configured to evaluate characteristic numbers for the vascular stiffness, such as the pulse wave velocities, as well as to perform the method according to the invention.

According to another aspect, the device for determining the central systolic blood pressure itself and/or the blood treatment machine may have a memory device, which is configured so that it can store the central systolic blood pressure determined according to the invention as well as optionally additional data measured or determined in conjunction with the determination of the central systolic blood pressure, such as the brachial systolic and diastolic blood pressure, the average blood pressure and/or a pulse curve, for example. The memory device may be designed to accommodate a memory medium on which the corresponding data is stored. Preferably one memory medium is used per patient. This may be a so-called patient card, for example. This ensures that patient-related data will remain on a memory medium assigned to the respective patient and will be stored there accordingly.

Additional advantages and embodiments of the invention are explained as an example on the basis of an exemplary embodiment with reference to the figures.

In these figures:

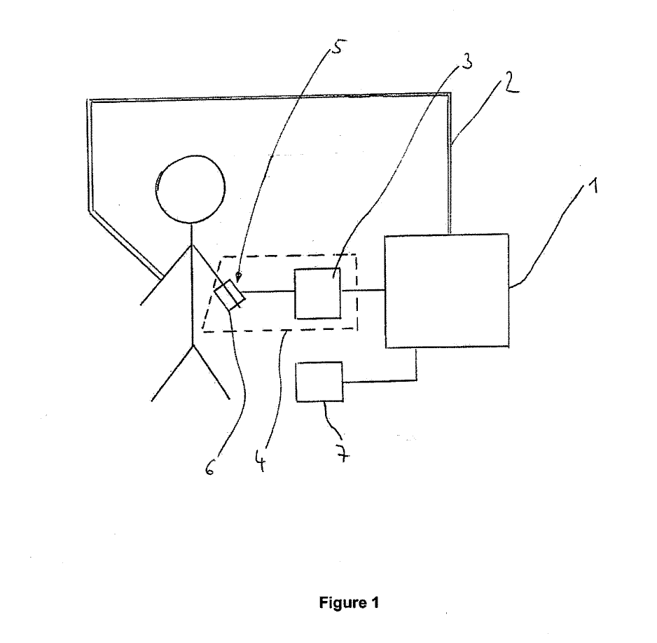

FIG. 1 shows schematically an arrangement of a device according to the invention for determining the central systolic blood pressure in combination with a blood treatment machine to which a patient is connected,

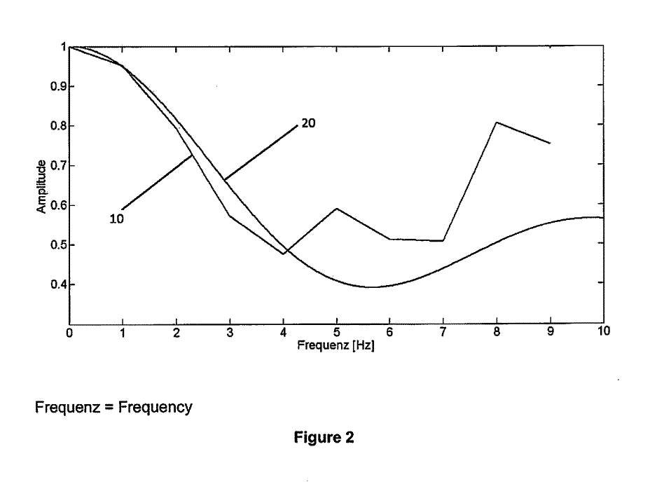

FIG. 2 shows a comparison of a transfer function, which is determined with a method according to the invention in comparison with a GTF (generalized transfer function) as a calibration transfer function, and

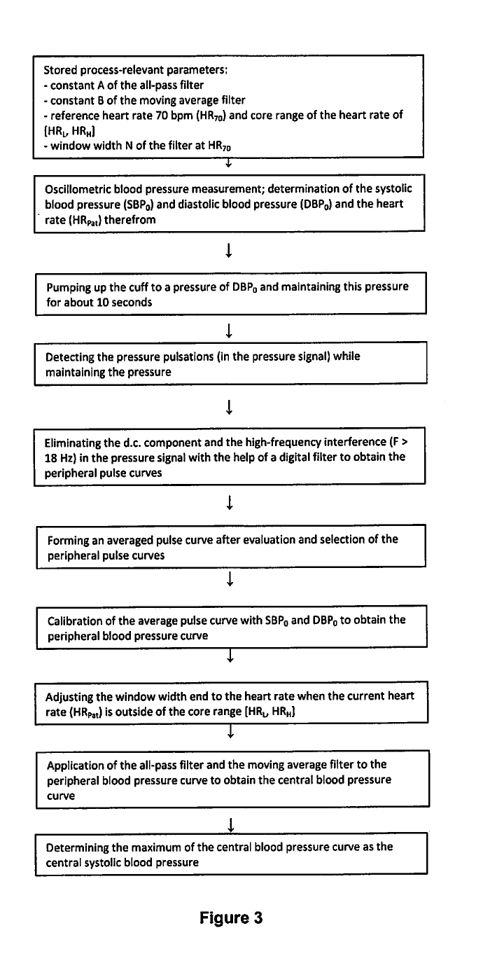

FIG. 3 shows a flowchart of an exemplary embodiment of the method according to the invention.

A blood treatment machine 1 according to the invention is suitable for performing dialysis, filtration and/or diafiltration, for example, in which a patient's blood is treated, sending it through bloodlines 2 into the blood treatment machine 1 and then back again. The blood treatment machine 1, as shown schematically in FIG. 1, has an evaluation unit 3, which in this exemplary embodiment is suitable for analyzing data that is directly associated with the blood treatment machine 1 and storing it as well as the data of a device 4 according to the invention for determining the central systolic blood pressure of the patient. With the evaluation unit 3, the machine 4 according to the invention determines a central systolic blood pressure as the maximum of a filtered blood pressure curve. The filtered blood pressure curve is obtained by the fact that the evaluation unit 3 applies an all-pass filter and a moving average filter to a peripheral blood pressure curve.

To do so, a peripheral blood pressure curve is obtained first. To do so, a sphygmomanometer having an inflatable cuff 6 that is placed on the patient's upper arm, is used as the blood pressure measuring device 5. Then a traditional oscillometric blood pressure measurement is performed and a diastolic blood pressure value, a systolic blood pressure value, an average blood pressure value and the pulse are obtained. The evaluation unit 3 is designed and configured to store these values. The scanning frequency f_{S}, with which the measured blood pressure values are recorded and/or stored, amounts to 500 Hz in this exemplary embodiment. The evaluation unit 3 has a unit that is also known as a “microcontroller” and must be equipped with only a low storage and computation capacity. In this case, it is an LPC2368 with 16/32 bits.

After the traditional blood pressure measurement, the cuff 6 is pumped up to the diastolic blood pressure, and the cuff pressure is maintained for approximately 10 seconds. During this period of time, the pressure measurement device 5 detects pressure oscillations, which are also known as “pulse curves.”

These pulse curves are first freed of the steady component and high-frequency interference (>18 Hz) and then stored in the evaluation unit 3 for further processing.

Unsuitable pulse curves are sorted out from the recorded pulse curves in advance, where these pulse curves include obvious outliers, as well as pulse curves that have been falsified due to movements by the patient, for example.

Then evaluation unit 3 forms an average pulse curve, which represents a peripheral blood pressure curve with which the additional process steps according to the invention are carried out.

The evaluation unit 3 calibrates the peripheral blood pressure curve with the systolic blood pressure curve and the diastolic blood pressure curve that are measured in advance. Thus, an absolute peripheral blood pressure curve is obtained for the patient and is stored temporarily by the evaluation unit 3.

The absolute peripheral blood pressure curve x(k) is filtered using the filter equation (1)

$$y ( k ) = A \cdot x ( k ) + B \cdot \frac{1}{2 m + 1} \cdot {\sum }_{i = - m}^{m} x ( k + i ) . \qquad ( 1 )$$

The filtered blood pressure curve y(k) is determined by the all-pass filter component A x(k), where A is an all-pass coefficient, and the average filter component is applied to the peripheral blood pressure curve, where B is the average coefficient and 2m+1 is the window width N of the time window, which is applied to the moving average value formation. It has been found that the window width with the amount of N=f_{S}/n yields the most accurate values for the central systolic blood pressure at a denominator n value of 4 when the heart rate is 70 bpm. A window width of 125 is obtained with f_{S}=500 Hz. With the preferred application of a symmetrical time window, the value 62 is obtained for m, because N=2m+1. Thus, in the present exemplary embodiment, a time window of 125 measured values, the average of each being formed, is plotted successively on the time axis of x(k), the absolute peripheral blood pressure curve of the patient. The resulting averages are multiplied times the coefficient of the moving average filter, also referred to as the “average coefficient,” and provided with the all-pass filter component. The evaluation unit determines the maximum as the central systolic blood pressure from this filtered blood pressure curve y(k), calculated by the evaluation unit 3, and stores it temporarily.

After the average curve has been determined over the respective time window of the window width N, the measured values that are no longer needed and intermediate calculations can be deleted or overwritten. This reduces the storage capacity burden on the microcontroller of the evaluation unit 3.

In this exemplary embodiment, the blood treatment machine optionally has a device 7 for determining the blood vessel stiffness; this device can be combined with the device for determining the central systolic blood pressure. The device for determining the blood vessel stiffness measures and evaluates the pulse wave velocity by pulse curve analysis as an indicator for vascular stiffness. This is also done in the evaluation unit 3 in the present case. Together with the vascular stiffness, the value of the central systolic blood pressure allows even more accurate information about cardiovascular diseases, if any. By combining the devices, an easy-to-operate overall unit that is highly compact is achieved with which these characteristic data are determined and detected with little effort during a blood treatment.

By approximation of the filter transfer function according to equation (2) to a calibration transfer function, for example, it is possible to determine A, B and N.

$$H ( f ) = A + \frac{B}{N} \cdot \frac{sin ( π Nf / {f}_{s} )}{sin ( π f / {f}_{s} )} \qquad ( 2 )$$

For example, a generalized transfer function determined on a patient population as reported by Y. T. Shih et al. (“Comparison of two generalized transfer functions for measuring central systolic blood pressure by an oscillometric blood pressure monitor” in: Journal of Human Hypertension 3, 204-210 (2013)) may be used as the calibration transfer function. FIG. 2 shows the inverted amplitude curve of this generalized transfer function as the curve labeled with reference numeral 10.

This yields, for example, a transfer function 20, such as that shown in FIG. 2, in addition to the calibration transfer function GTF at values of A=B=0.5, F_{S}=500 Hz, n=4, N=125. This shows the very good correspondence in the low frequency range with f≦4 Hz in particular.

The approximation described here need be performed only once for the patient. Therefore, it may be done in a separate unit of evaluation unit 3, which is not shown here but has a larger computation and storage capacity than the evaluation unit 3 itself. The approximation may also be performed on a completely separate computation unit, such as a personal computer. In the following sessions, the device 4 may be operated with stored values A, B, N, n, which are used in the filter equation (1), used for determining the central systolic pressure alone. The stored values may be retrieved by inserting a patient card into the evaluation unit 3, for example, which makes it possible to call up patient-specific data from the separate unit.

In the present exemplary embodiment, the patient population from which the calibration transfer function originates, had an average pulse of approximately 70 bpm, which corresponds to a heart rate of 1.17 Hz.

The patient in the exemplary embodiment also coincidentally has a pulse of 70 bpm in the measurement of the peripheral blood pressure curve that was performed. Therefore, a pulse-dependent correction of the described determination of the central systolic blood pressure is necessary.

However, in a second measurement of the peripheral blood pressure curve in another session for blood treatment using the blood treatment machine according to the invention, the patient has an elevated pulse of 90 bpm, which is thus outside of the predefined core range with a lower limit HR_{S} of 50 bpm and an upper limit HR_{H} of 85 bpm, so it is advantageous according to equation (3) to determine a pulse-corrected window width N₁, which is derived from the window width N:

$${N}_{1} = \frac{{f}_{s}}{nk} \cdot \frac{1}{{HR}_{pat} / {HR}_{Ref}} \qquad ( 3 )$$

The pulse-corrected window width N₁ of the quotient f_{S}/n₁ is therefore calculated, where n₁ is a pulse-corrected denominator. The pulse-corrected denominator is itself calculated by multiplying the denominator n from the determination of the window width N for the patient population that has a reference heart rate HR_{Ref} with a quotient HR_{Pat}/HR_{Ref}, where HR_{Pat} is the current heart rate of the patient in determination of the peripheral blood pressure, i.e. 90 bpm in the current case. The reference heart rate HR_{Ref} is the same heart rate as in the determination of the calibration pressure curve, i.e., 70 bpm here. This was calculated as the average heart rate of the patient population. Thus n₁ is calculated as 4×90/70=5.1. The corrected window width N₁ is obtained in turn at a scanning frequency of 500 Hz, rounded to the number of 98 measuring points in the time window.

According to one variant, the pulse-dependent denominator n₁ is again multiplied times a correction factor K, which permits a further adjustment of the filtered blood pressure curve to take into account additional factors such as medication, disease symptoms, etc. In the present case, K is assumed to be 1, so as not to perform a corresponding correction.

FIG. 3 shows as an example in one flowchart the sequence of the process according to the invention, where the values of A, B, N are already stored, in this case available in the memory. In this example, a heart rate of 70 bpm is selected as the reference heart rate HR_{Ref}. The lower limit HR_{S} of the core region is 50 bpm and the upper limit HR_{H} is 85 bpm.

All the steps just described may be performed by the blood treatment machine 1 as fully automatic or semiautomatic processes. They may also be performed with the device 4 for determining the central systolic blood pressure in a fully automatic or semiautomatic process, even without the blood treatment machine 1. In addition, they may also be performed with one or two of these devices by at least partial manual operation. However, the process described for this exemplary embodiment may also be carried out in principle with measurement equipment, controllers and/or regulators and other devices in addition to those explained here as examples.

1. A method for determining a patient's central systolic blood pressure, comprising the steps
determining a peripheral, in particular brachial, blood pressure curve,
applying a moving average filter to the blood pressure curve,
applying an all-pass filter to the blood pressure curve,
determining the central systolic blood pressure as the maximum of the all-pass-filtered and average-filtered blood pressure curve.

2. The method according to claim 1, characterized in that the peripheral blood pressure curve is determined in relative values, a systolic and a diastolic pressure of the patient are determined in absolute values and an absolute peripheral blood pressure curve is determined by calibration of the relative blood pressure curve with the systolic and diastolic blood pressure curves.

3. The method according to claim 1, characterized in that the patient's relative peripheral blood pressure curve, the systolic pressure, the diastolic pressure and/or the patient's pulse are all measured using a sphygmomanometer.

4. The method according to claim 1, characterized in that the moving average filter is applied in such a way that a fraction of the value of the scanning frequency (f_{S}) is selected as a window width (N) of a time window of a group of measuring points of the peripheral blood pressure curve for measuring the peripheral blood pressure curve, wherein the denominator (n) for forming the fraction is between 3 and 6, in particular between 3.8 and 4.2.

5. The method according to claim 4, characterized in that the window width (N) with the width N=2m+1 is applied to the time axis around the respective measuring point of the measured peripheral blood pressure curve, wherein m is an integral variable for defining a number of measuring points.

6. The method according to claim 1, characterized in that the all-pass filter is applied by adding an offset to the average-filtered blood pressure curve.

7. The method according to claim 5, characterized in that the filtered blood pressure curve (y(k)) is determined according to a first filter equation (equation 1):
$$y ( k ) = A \cdot x ( k ) + B \cdot \frac{1}{2 m + 1} \cdot {\sum }_{i = - m}^{m} x ( k + i )$$
or according to a second filter equation (equation 4):
$$y ( k ) = A \cdot x ( k ) + B \cdot \frac{1}{2 m + 1} \cdot {\sum }_{i = - m}^{m} x ( k + i - ϕ )$$
where A is an all-pass coefficient and B is an average filter coefficient, where A+B=1, x(k) corresponds to the absolute peripheral blood pressure curve, k corresponds to the index of the measuring points of the peripheral blood pressure curve and i corresponds to the index of neighboring measuring points for determination of the average and also φ corresponds to a phase angle.

8. The method according to claim 7, characterized in that the all-pass coefficient (A), the average filter coefficient (B), the window width (N) and/or the denominator (n) are determined by the fact that a transfer function (H(f)) of a calibration transfer function that corresponds to the filter equation (equation 1) is approximated to a calibration transfer function.

9. The method according to claim 8, characterized in that a certain generalized transfer function determined on a patient population is used as the calibration transfer function.

10. The method according to claim 9, characterized in that the window width (N) is corrected with a pulse-dependent correction factor to yield a pulse-corrected window width (N₁).

11. The method according to claim 10, characterized in that the pulse-corrected window width (N₁) is calculated by a correction equation (equation 3), according to which:
$$N = \frac{\vert {f}_{s} \vert}{nk} \cdot \frac{1}{{HR}_{pat} / {HR}_{Ref}}$$
where f_{S} corresponds to the sampling frequency in hertz, n is the denominator, k is a correction factor, HR_{Pat} is the current heart rate of the patient and HR_{Ref} is the reference heart rate.

12. A device for determining the central systolic blood pressure of a patient, comprising a blood pressure measuring device for determining a peripheral, in particular brachial, blood pressure curve and an evaluation unit which is configured for
applying a moving average filter to the blood pressure curve,
applying an all-pass filter to the blood pressure curve,
determining the central systolic blood pressure as a maximum of the filtered blood pressure curve.

13. The device according to claim 12, characterized in that the blood pressure measurement device is suitable for determining the peripheral blood pressure curve as a relative value and for determining a systolic pressure and a diastolic pressure on a patient as absolute values, and the evaluation unit is configured to determine an absolute peripheral blood pressure curve by calibration of the relative blood pressure curve with the absolute systolic and diastolic pressure values.

14. The device according to claim 12, characterized in that the evaluation unit is configured to perform a method of determining a patient's central systolic blood pressure, comprising the steps
determining a peripheral, in particular brachial, blood pressure curve,
applying a moving average filter to the blood pressure curve,
applying an all-pass filter to the blood pressure curve, and
determining the central systolic blood pressure as the maximum of the all-pass-filtered and average-filtered blood pressure curve.
wherein the moving average filter is applied in such a way that a fraction of the value of the scanning frequency (f_{S}) is selected as a window width (N) of a time window of a group of measuring points of the peripheral blood pressure curve for measuring the peripheral blood pressure curve, wherein the denominator (n) for forming the fraction is between 3 and 6, in particular between 3.8 and 4.2

15. A blood treatment machine having an extracorporeal blood circulation for treatment of a patient's blood, comprising a device for determining the central systolic blood pressure of the patient according to claim 11.
